(19) United States
(12) Patent Application Publication
(10) Pub. No.: US 2019/0298908 A1
(43) Pub. Date: Oct. 3, 2019

(54) Process and Device for Depleting a Targeted Substance and Use Thereof

(71) Applicants: Yan Chen, (US); Gang G. Xu, Wilmington, DE (US); Juanjuan Jiang, (US); Juan Sheng, (US)

(72) Inventors: Yan Chen, Nanjing (CN); Gang G. Xu, Wilmington, DE (US); Juanjuan Jiang, Nanjing (CN); Juan Sheng, Nanjing (CN)

(21) Appl. No.: 16/438,437

(22) Filed: Jun. 11, 2019

Related U.S. Application Data
(60) Provisional application No. 62/686,081, filed on Jun. 17, 2018.

(51) Int. Cl. A61M 1/36; A61M 1/34

(52) U.S. Cl. CPC A61M 1/362; A61M 1/3679; A61M 1/3486

(57) ABSTRACT

This invention is directed to a process for depleting one or more targeted substances such as toxins, proteins such as one or more immune checkpoint inhibitors (ICI), or a combination thereof from a subject. Immune checkpoint inhibitors (ICI), such as PD-1 protein, PD-1 ligand (PD-L1), CTLA-4, LAG-3, TIM3 and others are known to be involved in inhibition of T-cell activation in tumor patients. This invention is also directed to a device for depleting ICI such as PD-1 protein, PD-1 ligand (PD-L1) including soluble PD-L1 (sPD-L1), PD-L2, CTLA-4 including soluble form sCTLA-4, LAG-3, TIM3, or toxins from a subject. This invention is further directed to the use of the process and the devices for treating medical conditions such as cancers, other diseases and substance overdose.

### CROSS REFERENCE

This application claims the priority of U.S. provisional application Ser. No. 62/686,081 filed on Jun. 17, 2018, which is hereby incorporated by reference.

### FIELD OF THE INVENTION

This invention is directed to a process and a device for depleting at least one target substance from a body fluid of a subject for treating a medical condition.

### BACKGROUND

Immunotherapy has emerged as a promising approach for fighting against cancer. Monoclonal antibodies (mAb) against PD-1, such as Keytruda (pembrolizumab) and Opdivo (nivolumab), against PD-L1, such as Bavencio (avelumab), Imfinzi (durvalumab), Tecentriq (atezolizumab) and against CTLA-4 such as Yervoy (Ipilimumab) have been successfully used in cancer treatments in many patients.

Different from chemotherapy and small molecule inhibitors, these antibodies reactivate anti-tumor immune responses of immune cells through blockades of immune checkpoint inhibitors (ICI), such as PD-1 protein (programmed cell death protein 1) and PD-1 ligand (PD-L1) and associated signaling pathways. Antibodies against Checkpoint inhibitors can block tumor induced inhibition of T-cell activation in tumor patients. It is currently recognized that generation of tumor-reactive T cells (typically CD28 T cells) requires tumor-associated antigens being presented by antigen-presenting cells (APCs, also known as dendritic cells) and also involving MHC I/II (Major histocompatibility complex I/II). A T-cell receptor (TCR) recognizes MHC-bound tumor antigen providing an initial signal for T-cell activation. Full T-cell activation will follow after the engagement of additional molecules. The activated T cells then undergo differentiation, expansion and ultimately releasing cytolytic effector molecules killing tumor cells. Tumor cells, however, can evade such T-cell responses by producing a number of molecules to inhibit the recognition by T cells and subsequent activation of the T cells. PD-L1 is one of such molecules that can bind to PD-1 producing an inhibitory signal reducing the proliferation of antigen-specific T-cells. Blockade of the PD-L1 binding to PD-1 by an antibody is believed to prevent such tumor induced inhibition of T-cell activation.

Recent studies, however, show that these antibodies can also cause some side effects such as renal toxicity, renal disfunction including acute kidney injury (AKI) at a high rate ranging from 9% to 29% (Wanchoo, et al., American Journal of Nephrology, 45:160-169, 2017) and autoimmune disease such as type 1 diabetes in adenocarcinoma of the lung and sarcomatoid squamous cell carcinoma (Mellati, et al., Diabetes Care, 38 (9): e137-e138, 2015). Long term use, such as 6 to 12 months, of PD-1/PD-L1 antibodies showed significant rate of AKI. Some patients may also show hematuria, eosinophilia and worsening hypertension. Majority of patients suffering from PD-1/PD-L1 immunotherapy related AKI exhibit rising serum creatinine and pyuria.

Therefore, there is a need for a new process for improving immunotherapy or providing a new or alternative treatment for cancer.

### SUMMARY

The present invention is directed to a process for depleting at least one target substance from a body fluid of a subject. The process comprises the steps of: directing the body fluid of the subject into a capturing device, wherein the body fluid contains the target substance, and wherein the capturing device comprises an affinity matrix comprising an immobile phase and at least one affinity agent affixed to the immobile phase, the affinity agent is configured to bind to the target substance; and passing the body fluid through the affinity matrix for the affinity agent to bind and immobilize the target substance to the immobile phase, producing a processed body fluid having at least a portion of the target substance depleted therefrom.

The present invention is also directed to a capturing device for depleting at least one target substance from a body fluid. The device comprises: an affinity matrix having an immobile phase having at least one affinity agent affixed thereon, the affinity agent is configured to bind to the target substance and immobilize the target substance on the immobile phase; and a device housing to house the affinity matrix, the device housing comprises a housing intake for receiving the body fluid into the device housing, a fluid passage for containing the affinity matrix and a housing outlet, the fluid passage is functionally coupled to the housing intake and the housing outlet; wherein the capturing device is configured to receive the body fluid through the housing intake, pass the body fluid through the fluid passage and output a processed the body fluid from the housing outlet.

The present invention is also directed to a method for treating a medical condition in a subject in need thereof. The method comprises: directing a body fluid of the subject into a capturing device, wherein the body fluid contains at least one target substance related to the medical condition, and wherein the capturing device comprises an immobile phase and at least one affinity agent affixed to the immobile phase, the affinity agent is configured to bind to the target substance; passing the body fluid through the capturing device for the affinity agent to bind to the target substance and immobilize at least a portion of the target substance on the immobile phase, producing a processed body fluid having at least a portion of the target substance removed therefrom; and directing the processed body fluid back to the subject.

### BRIEF DESCRIPTION OF THE FIGURES

FIG. 1A-FIG. 1B. An example of a schematic illustration of a device (A) and immobilized affinity agent (B).

FIG. 2A-FIG. 2D. Examples of schematic illustrations of a device (A) and immobilized phase containing multiple affinity agents (B) and individual affinity agents (C) and (D).

FIG. 3A-FIG. 3D. Examples of schematic illustrations of fusion proteins. (A) A carrier polypeptide linked to a target binding polypeptide with the carrier at the N-terminal of the fusion protein. (B) A carrier polypeptide linked to multiple target binding polypeptides with the carrier at the N-terminal of the fusion protein. (C) A carrier polypeptide linked to a target binding polypeptide with the carrier at the C-terminal of the fusion protein. (D) A carrier polypeptide linked to multiple target binding polypeptides with the carrier at the C-terminal of the fusion protein.

FIG. 4A-FIG. 4C. Examples of fusion proteins with one or more linkers. (A) A carrier linked to a linker and then linked to a target binding polypeptide with the carrier at N-terminal of the fusion protein. (B) A carrier polypeptide linked to a linker and then multiple target binding polypeptides. (C) A carrier polypeptide linked to a first linker then linked to a first target binding polypeptide with a subsequent linker in between the first and the second target binding polypeptides.

FIG. 5. A schematic illustration of an example of a system configuration with body fluid flowing through a capturing device.

FIG. 6. A schematic illustration of an example of a system configuration with body fluid flowing through a capturing device and a dialysis device in a tandem configuration.

FIG. 7. A schematic illustration of an example of a system configuration with body fluid flowing through a capturing device and a dialysis device in a parallel configuration.

FIG. 8. A schematic illustration of an example of an application using the capturing device.

FIG. 9A-FIG. 9B. Schematic illustrations of examples of a capturing-dialysis hybrid device. (A) A capturing device and a dialysis device share a common intake to receive the body fluid flowing first through the capturing device. (B) A capturing device and a dialysis device share a common outlet to output a processed body fluid.

### DETAILED DESCRIPTION

Features and advantages of the present invention will be more readily understood, by those of ordinary skill in the art, from reading the following detailed description. It is to be appreciated that certain features of the invention, which are described above and below in the context of separate embodiments, may also be provided in combination in a single embodiment. Conversely, various features of the invention that are, for brevity, described in the context of a single embodiment, may also be provided separately or in any combination or sub-combination. In addition, references in the singular may also include the plural (for example, “a” and “an” may refer to one, or one or more) unless the context specifically states otherwise.

Use of numerical values in the various ranges specified in this application, unless expressly indicated otherwise, are stated as approximations as though minimum and maximum values within the stated ranges were both proceeded by the word, “about”. In this manner, slight variations above and below the stated ranges can be used to achieve substantially the same results as values within the ranges. Also, disclosure of ranges is intended as a continuous range including every value between the minimum and maximum values and including the minimum and maximum cited values.

The term “body fluid”, “body fluids”, “bodily fluid” or “bodily fluids” refers to liquids within the body of a living person or animal. This body fluid can comprise intracellular fluid (ICF), extracellular fluid (ECF) or a combination thereof. The ECF can be divided into the fluid between cells, the “interstitial fluid” and the “vascular fluid” that can include the blood plasma. The vascular fluid can be further divided into the venous fluid, such as blood in veins and the arterial fluid, such as blood in artery. Examples of body fluid can include, but not limited to, amniotic fluid, aqueous humour, vitreous humour (both are fluids in eyes), bile, blood, blood plasma, blood serum, breast milk, cerebrospinal fluid, cerumen (earwax), chyle, chime, endolymph and perilymph, exudates, gastric juice, lymph, mucus (including nasal drainage and phlegm), pericardial fluid, peritoneal fluid, pleural fluid, rheum, saliva, semen, sputum, synovial fluid, sweat, tears, urine, and so on. A blood plasma containing no clotting factors is often referred to as a blood serum.

The term “depletion”, “depleting”, “depleted” or other grammatical variations used herein means a partial or a complete removal of a target substance from a body fluid. Depletion of a target substance can be in a range of from 0.001% to 100%, such as 0.001% to 100% in one example, 0.1% to 100% in another example, 1% to 100% in yet another example, 10% to 100% in yet another example, 20% to 100% in yet another example, 30% to 100% in yet another example, 40% to 100% in yet another example, 50% to 100% in yet another example, 60% to 100% in yet another example, 70% to 100% in yet another example, 80% to 100% in yet another example, 90% to 100% in yet another example, 95% to 100% in yet a further example, 0.001% to 90% in yet another example, 0.001% to 80% in yet another example, 0.001% to 70% in yet another example, 0.001% to 60% in yet another example, 0.001% to 50% in yet example, 0.001% to 40% in yet another example, 0.001% to 30% in yet another example, 0.001% to 20% in yet another example, 0.001% to 10% in yet another example and 0.001% to 5% in a further example. Percentage used herein is based on the targeted substance measured before and after the depletion using a same measurement method. For example, a 90% depletion of a target substance means 90% of the target substance is depleted from the body fluid after the depletion process leaving about 10% of the target substance remaining in the body fluid based on a same measurement before and after the depletion process. The measurement can be quantitative or qualitative and can be based on weight, volume, molar unit, activity, bioreactivity, reaction to a marker or one or more pre-determined markers, or a combination thereof. In one example, plasma level of PD-1 protein can be measured before and after the depletion process using a PD-1 ELISA kit commercially available. In another example, PD-L1 level in serum can be determined using a PD-L1 ELISA kit commercially available. In yet another example, PD-1, PD-L1 or PD-L2 protein levels in blood can be determined with Western Blot by reacting with the anti-PD1, anti-PD-L1 or anti-PD-L2 antibodies, respectively. In yet another example, protein levels of a target protein can be determined using ELISA method using an antibody specific to the target protein. In yet an example, amounts of particles of a targeted substance in a body fluid can be determined by visual inspection or optical measurement.

The term “CTLA-4” refers to the protein Cytotoxic T-Lymphocyte-associated Antigen 4, a soluble form of CTLA-4, a gene or mRNA encoding the same. A soluble form, referred to as sCTLA-4, is generated form an alternatively spliced mRNA. Low levels of sCTLA-4 are detected in normal human serum. CTLA-4 is implicated in T cell activation. Increased serum levels of CTLA-4 are observed in some autoimmune diseases. The term “PD-1” refers to the Programmed death-1 protein or a gene encoding the same. The term “PD-L1” refers to the ligand of PD-1 protein or the gene encoding the same. The term “LAG3” refers to lymphocyte activation gene 3 or a gene encoding the same. The term “TIM3” refers to T cell membrane protein 3, also known as T-cell Immunoglobulin domain and Mucin domain 3 or a gene encoding the same. For brevity, each of the protein names can refer to a soluble protein, a membrane-bound protein, or both, unless specifically defined.

The term “antibody” or “antibodies” refers to a polyclonal antibody, a monoclonal antibody (mAb), unless specifically defined. An antibody can be of any immunoglobin subtypes, such as immunoglobulin A (IgA), immunoglobulin G (IgG), immunoglobulin M (IgM), immunoglobulin D (IgD), immunoglobulin E (IgE), or a combination thereof, unless specifically defined. An antibody can also be a hybrid antibody that combines molecular components of two or more antibodies, a recombinant antibody that is produced from recombinant genes or synthesized polypeptides.

This invention is directed to a process for depleting at least one target substance from a body fluid of a subject, the process comprising the steps of:

directing the body fluid of the subject into a capturing device, wherein the body fluid contains the target substance, and wherein the capturing device comprises an affinity matrix comprising an immobile phase and at least one affinity agent affixed to the immobile phase, the affinity agent is configured to bind to the target substance; and

passing the body fluid through the affinity matrix for the affinity agent to bind and immobilize the target substance to the immobile phase, producing a processed body fluid having at least a portion of the target substance depleted therefrom.

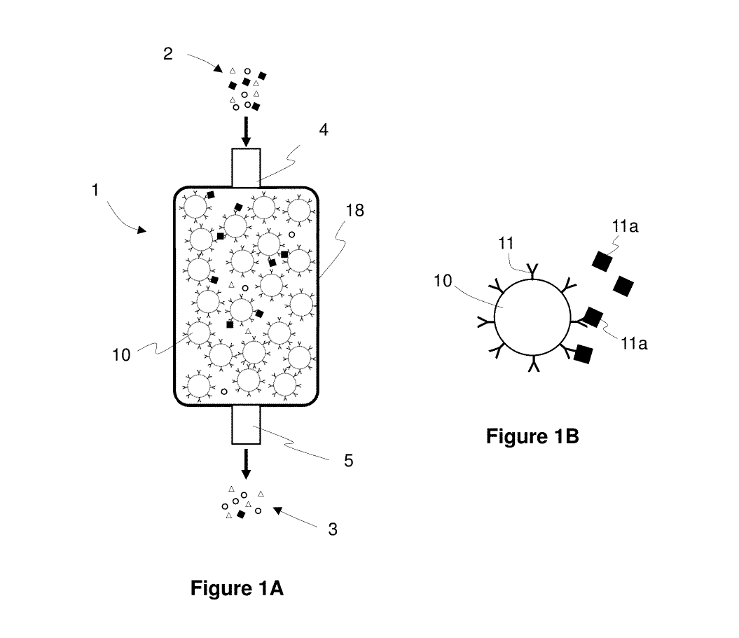

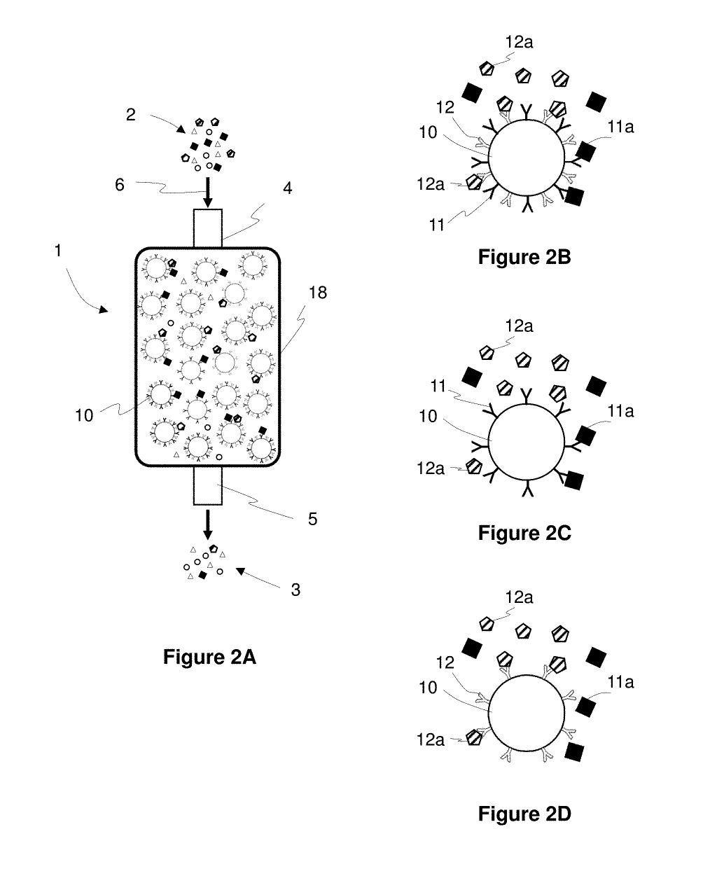

A device (1) exemplified in FIG. 1-FIG. 2 can be suitable for the process of this invention. A body fluid (2) comprising one or more target substances can be directed to the device (6) and the one or more target substances (11a-12a) can be bound to corresponding affinity agents (11-12) affixed to the immobile phase (10) to produce a processed body fluid (3).

The affinity agent can comprise a biological affinity agent, a chemical affinity agent, a physical affinity agent, or a combination thereof, wherein the biological affinity agent can comprise a protein, an antibody, a binding fragment of an antibody, an antigen, a fragment of an antigen, a neoantigen polypeptide comprising one or more neoantigens or epitopes, an antagonist, a receptor, nucleotides, deoxynucleotides, thiolated nucleotides, a binding fragment of a receptor, a bacteria-binding protein, a virus-binding protein, an opioid receptor, a toxin-binding protein, a fusion protein comprising a target binding polypeptide linked to a carrier, or a combination thereof.

The affinity agent can be one member of a binding pair, such as an antibody or an antigen in an antibody-antigen binding pair, a ligand or a receptor in a ligand-receptor binding pair, a substrate or an enzyme in a substrate-enzyme binding pair, a protein or a nucleotide (RNA) in a protein-RNA binding pair, a protein or a deoxynucleotide (DNA) in a protein-DNA binding pair, a biotin or an avidin in a biotin-avidin binding pair, a biotin or a streptavidin in a biotin-streptavidin binding pair, an opioid or an opioid receptor in an opioid-receptor binding pair, a DNA in a DNA-DNA binding pair such as matching strands of a double strand DNA, a DNA or a RNA in a DNA-RNA binding pair, and so on. The affinity agent can also be a chemical affinity agent, such as an ion exchange resin or beads that can bind to a substrate based on charges, chemical groups, chelation, or others. In one example, the affinity agent can be an antibody, an antigen, a fragment thereof, or a combination thereof.

The thiolated nucleotides can comprise thio-bounds between nucleotides instead of phosphate bounds. A thiolated nucleotides is generally more stable than the native phosphate bound containing nucleotides.

By affixing any of the aforementioned affinity agent, such as a member of a binding pair, to an immobile phase, the affinity agent can be configured to bind to the target substance, such as the other member of a binding pair, from blood or other body fluid and immobilize the target substance to the immobile phase, therefore depleting the target substance from the body fluid.

The antibody can comprise an anti-PD-1 antibody, an anti-PD-L1 antibody, an anti-PD-L2 antibody, an anti-CTLA-4 antibody, an anti-LAG-3 antibody, an anti-TIM3 antibody or a combination thereof. The antibody can also comprise an antibody against a small molecule, wherein the antibody is produced by linking the small molecule to a protein carrier, for example, an antibody against a nerve agent, a pesticide such as myclobutanil, opioid, tetrahydrocannabinol (THC), cannabinol or cannabinodiol (CBD). The protein carrier can be HSA or other proteins have sufficient molecular weight, such as greater than 10 KDa, and low antigenicity.

The fusion protein can comprise one or more target binding polypeptides linked to a carrier. The target binding polypeptide can be protein, an antibody, a binding fragment of an antibody, an antigen, a fragment of an antigen, a neoantigen polypeptide comprising one or more neoantigens or epitopes, an antagonist, a receptor, a binding fragment of a receptor, a bacteria-binding protein, a virus-binding protein, a toxin-binding protein, or a combination thereof. The fusion protein can comprise one or more of target binding polypeptides selected from a PD-1 binding polypeptide, a PD-L1 binding polypeptide, a PD-L2 binding polypeptide, a CTLA-4 binding polypeptide, a LAG-3 binding polypeptide, a TIM3 binding polypeptide, a checkpoint inhibitor binding polypeptide, a neoantigen polypeptide, or a combination thereof. In one example, the fusion protein can comprise two or more polypeptides selected from a PD-1 binding polypeptide and at least one of PD-L1 binding polypeptide, PD-L2 binding polypeptide, CTLA-4 binding polypeptide, LAG-3 binding polypeptide, TIM3 binding polypeptide, a checkpoint inhibitor binding polypeptide, or a neoantigen polypeptide. In another example, the fusion protein can comprise two or more polypeptides selected from a PD-L1 binding polypeptide and at least one of PD-1 binding polypeptide, PD-L2 binding polypeptide, CTLA-4 binding polypeptide, LAG-3 binding polypeptide, TIM3 binding polypeptide, a checkpoint inhibitor binding polypeptide or a neoantigen polypeptide. The term “a checkpoint inhibitor binding polypeptide” or “another checkpoint inhibitor binding polypeptide” refers to a polypeptide that binds to the same ICI such as PD-1, PD-L1, CTLA-4, LAG-3 or TIM3 at a different binding domain, or a protein that is different from PD-1, PD-L1, CTLA-4, LAG-3 or TIM3, but has an immune checkpoint inhibitor function. In yet another example, a fusion protein can comprise one or more PD-L1 binding polypeptides and one or more PD-1 binding polypeptides. The one or more PD-1 binding polypeptides can have same or different binding sites on the PD-1 protein. The one or more PD-L1 binding polypeptides can have same or different binding sites on the PD-L1 protein.

The carrier can comprise a human serum albumin (HSA) or a functional variant thereof. Suitable carrier can comprise HSA purified from human blood, produced by chemical synthesize, produced by recombinant gene expressed in a host, or a combination thereof. A functional variant can comprise a fragment of any of the aforementioned HSA, an HSA containing one or more mutations, modification, or a combination thereof. The carrier can also comprise a bovine serum albumin (BSA), a variant thereof or other suitable polypeptides. The carrier can be selected from a polypeptide having low immunogenicity or antigenicity. The carrier can also be modified to have low immunogenicity or antigenicity via mutations or replacement of certain amino acid residues. Not wishing to be bound by a particular theory or mechanism, it is believed that a carrier can help to stabilize the target binding polypeptide, better present the target binding polypeptide for improved binding with a target substance, or a combination thereof.

In the process of this invention disclosed herein, the fusion protein can be produced by a production process comprising expressing a fusion coding region comprising codes encoding the target binding polypeptide and the carrier in an expression host. The expressed fusion protein can be purified or modified including but not limited to refolding, glycosylation, or a combination thereof. The expression host can be bacteria cells, yeast cells, plant cells, eggs, mammalian cells, cell lines, cell free expression systems, or a combination thereof. Expression vectors can be used to construct and express the fusion protein. In addition to the coding region (also known as an open reading frame), suitable expression vectors can comprise an expression cassette comprising an eukaryotic promoter, a prokaryotic promoter, an enhancer, a terminator, a 3′ untranslated region such as a polyA tail region, or a combination thereof. A constitutive or an inducible promoter can be suitable. Other sequences, such as a signal sequence, can also be included in an expression vector.

The fusion protein can comprise one or more linker peptides positioned between the target binding polypeptide and the carrier, between two target binding polypeptides, or a combination thereof.

The fusion protein can further comprise at least one coupling moiety, such as His-tag, glutathione-GST affinity moiety, or a combination thereof, for easy immobilization of the fusion protein to an immobile phase.

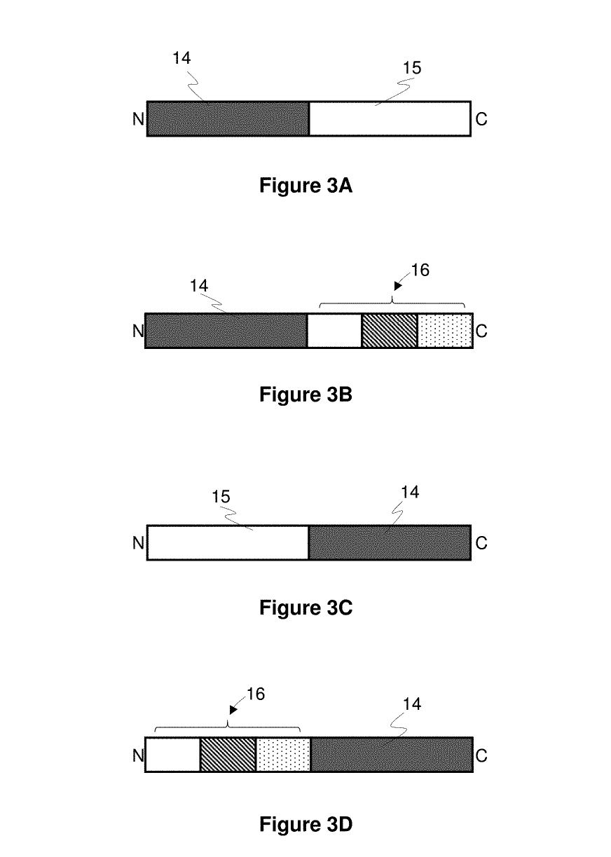

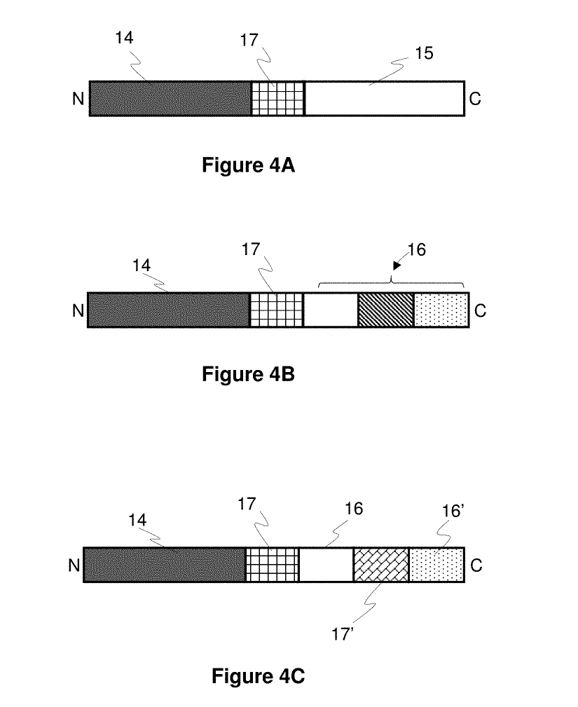

The fusion protein can be configured to be a single polypeptide having a carrier (14) at its N-terminal portion and one or more target binding polypeptides (15 and 16) at its C-terminal portion (FIG. 3A and FIG. 3B). The fusion protein can also be configured to be a single polypeptide having a carrier (14) at its C-terminal portion and one or more target binding polypeptides (15 and 16) at its N-terminal portion (FIG. 3C and FIG. 3D). The fusion protein can also be configured to be a single polypeptide having a carrier at middle portion, one or more target binding polypeptides at its N-terminal portion and additional one or more target binding polypeptides at its C-terminal portion. A linker peptide (17 or 17′) can be positioned between the carrier (14) and one or more target binding polypeptides (15 or 16) or between two target binding polypeptides (16 and 16′) (FIG. 4A-FIG. 4C). A linker peptide can comprise in range of from 0 to 100 amino acid residues in on example, 0 to 80 amino acid residues in another example or 0 to 50 amino acid residues in yet another example.

One advantage of the fusion protein can include lower production cost, consistent and reproducible polypeptide structure, such as consistent binding structure, etc., compared to polyclonal or monoclonal antibodies that may have high potential with batch to batch variations.

The target substance can have a molecular weight in a range of from 5 to 5,000 KDa (kilodaltons). The target substance can have a molecular weight in a range of from 5 to 5,000 KDa in one example, 5 to 500 KDa in another example, 5 to 100 KDa in yet another example, 10 to 5,000 KDa in a further example, 10 to 500 KDa in yet a further example and 10 to 100 KDa in an even further example. In additional example, the target substance can comprise proteins such as PD-1 (about 40 KDa), PD-L1 (about 20 KDa), PD-L2, CTLA-4 (about 50 KDa). In another example, the target substance can comprise cells, bacteria or viral particles that can be in a range of from 500-5,000 KDa. In a further example, the target substance can have a molecular weight (MW) of greater than 10 KDa. In yet a further example, the target substance can have a molecular weight greater than the molecular weight pore size limits of conventional hemodialysis membranes, which typically have pore size at a MW cut-off in a range of from 3 KDa to 15 KDa. Typically, substances that have MW less than the molecular weight pore size limits of conventional hemodialysis membranes can be easily removed from hemodialysis.

In process of this invention disclosed herein, the target substance can comprise a protein, a chemical agent, a toxin, a drug, bacteria, a virus, virus nucleic acids, virus protein, a particle, an aggregate or a combination thereof. The target substance can comprise a DNA, RNA, virus DNA, virus RNA, DNA-protein complex, RNA-protein complex, single strand DNA, single strand RNA, DNA-RNA complex, protein-protein complex, protein-membrane complex, protein-lipid complex, complex of protein or nucleic acid with carbohydrates, or a combination thereof. The target substance can also comprise chemical agent including drugs, toxic substances, toxins, etc. The target substance can be hydrophilic and soluble in aqueous solutions, such as body fluid, water, saline, aqueous buffer, or a combination thereof.

The body fluid can be aqueous humour, vitreous humour, bile, blood, blood plasma, blood serum, gastric juice, lymph or a combination thereof. In one example, a body fluid comprises blood or a portion thereof. In another example, a body fluid comprises aqueous humour and vitreous humour, fluids in eyes.

The process of this invention disclosed herein can further comprise directing the body fluid through a dialysis device selected from a hemodialysis device or a peritoneal dialysis device, wherein the body fluid is directed to flow through the capturing device and the dialysis device tandemly or parallelly. The body fluid can be directed at a flow rate in a range of from 1 ml/min to 250 ml/min passing through the capturing device.

The process can further comprise the step of directing the processed body fluid back to the subject.

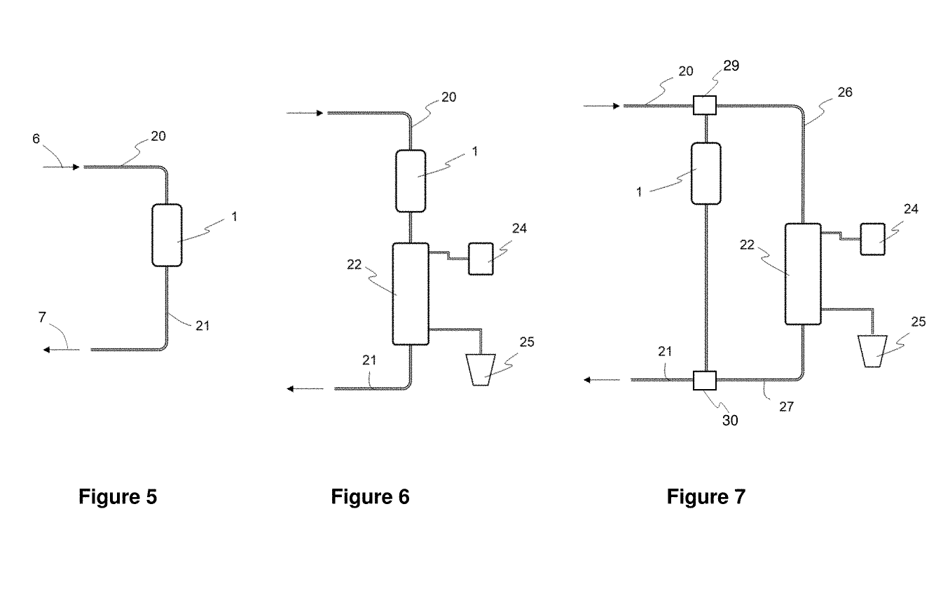

System configurations exemplified in FIG. 5-FIG. 7 can be suitable. For example, a capturing device (1) can be used alone, wherein a body fluid can be directed in an input direction (6) through an input connection coupling (20) to the capturing device (1), then output in an output direction (7) through an output connection coupling (21) (FIG. 5). A capturing device (1) can be tandemly coupled to a dialysis device (22) that is functionally coupled to a dialysis fluid storage device (24) and a dialysis drainage device (25) (FIG. 6). A capturing device (1) can also be parallelly coupled to a dialysis device (22) (FIG. 7). In this parallel configuration, the body fluid can be split via an input splitting device (29) to direct part of the body fluid to the capturing device and part of the fluid to the dialysis device via a dialysis input coupling (26). After dialysis, the body fluid can be joined together via a dialysis output coupling (27) and a joining device (30) (FIG. 7), before returning to the subject.

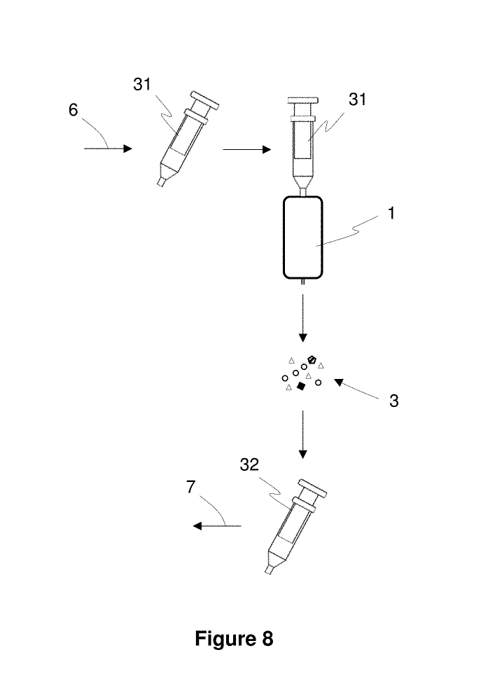

The body fluid can also be directed to an input container (31), such as a syringe. The input container can then be coupled to a capturing device (1) to pass the body fluid through it producing a processed body fluid (3) (FIG. 8). The processed body fluid can be collected in a delivery device (32), such as a second syringe and directed back to the patient, for example.

The process of this invention can further comprise measuring a pre-concentration of the target substance in the body fluid before passing through the capturing device, a post-concentration of the target substance in the processed body fluid, or a combination thereof.

This invention is also directed to a capturing device for depleting at least one target substance from a body fluid, the device comprising:

an affinity matrix having an immobile phase having at least one affinity agent affixed thereon, the affinity agent is configured to bind to the target substance and immobilize the target substance on the immobile phase; and

a device housing to house the affinity matrix, the device housing comprises a housing intake for receiving the body fluid into the device housing, a fluid passage for containing the affinity matrix and a housing outlet, the fluid passage is functionally coupled to the housing intake and the housing outlet;

wherein the capturing device is configured to receive the body fluid through the housing intake, pass the body fluid through the fluid passage and output a processed the body fluid from the housing outlet.

A capturing device exemplified in FIG. 1A and FIG. 2A can be suitable. The device housing (18) can comprise a housing intake (4) and a housing outlet (5) (FIG. 1A and FIG. 2A). The device housing can be flexible or rigid. In one example, the device housing can be a flexible plastic bag. In another example, the device housing can be a tube or a column. The immobile phase (10) can comprise one affinity agent, such as a first antibody (11) that is configured to bind to a first target substance (11a) (FIG. 1B). The immobile phase (10) can also comprise two or more affinity agents, such as a first antibody (11) that is configured to bind to a first target substance (11a) and a second antibody that is configured to bind to a second target substance (12a) (FIG. 2B-FIG. 2D). The capturing device is configured to allow the body fluid to interact with the affinity agent in the capturing device to immobilize the target substance to the immobile phase so that the process body fluid can comprise reduce amount of the target substance or be depleted from the target substance.

The immobile phase can be selected from beads, membrane, porous matrix, scaffold, or a combination thereof. Affinity agent can be immobilized or “coupled” directly to solid support material (immobile phase) by formation of covalent chemical bonds between one or more functional groups on the affinity agent, such as primary amines, sulfhydryls, carboxylic acids, aldehydes, etc., and reactive groups on the immobile phase. The immobile phase can comprise agarose, Cellulose, Protein A, silicon, or a combination thereof. Specific coupling moieties, such as a glutathione S-transferase (GST)-tagged fusion protein can be first captured to a glutathione support via the glutathione-GST affinity interaction and then secondarily chemically crosslinked to immobilize it. In another example, a His-tag can be affixed to an immobile phase and then a fusion protein comprises a His-tag can be reacted to the His-tag and immobilized. Commercial available beads and other matrixes suitable for immobile phase can be used. The immobile phase remains stationary when the body fluid is passing through the affinity matrix of the capturing device. Such stationary operation is advantageous since it eliminates the risk of damaging cells, such as blood cells during the depletion operation.

The affinity agent can comprise a biological affinity agent, a chemical affinity agent, a physical affinity agent, or a combination thereof, wherein the biological affinity agent can comprise a protein, an antibody, a binding fragment of an antibody, an antigen, a fragment of an antigen, a neoantigen polypeptide comprising one or more neoantigens or epitopes, an antagonist, a receptor, nucleotides, deoxynucleotides, thiolated nucleotides, a binding fragment of a receptor, a bacteria-binding protein, a virus-binding protein, an opioid receptor, a toxin-binding protein, a fusion protein comprising a target binding polypeptide linked to a carrier, or a combination thereof. Any of aforementioned affinity agents can be suitable. The antibody can comprise an anti-PD-1 antibody, an anti-PD-L1 antibody, an anti-PD-L2 antibody, an anti-CTLA-4 antibody, an anti-LAG-3 antibody, an anti-TIM3 antibody, or a combination thereof, as disclosed above and hereafter.

For the capturing device disclosed herein, the fusion protein can be produced by a production process comprising expressing a fusion coding region comprising codes encoding the target binding polypeptide and the carrier in an expression host, as described above and hereafter.

For the capturing device of this invention, aforementioned target binding polypeptides can be suitable. For example, the target binding polypeptide can comprise a PD-1 binding polypeptide, a PD-L1 binding polypeptide, a PD-L2 binding polypeptide, a CTLA-4 binding polypeptide, a LAG-3 binding polypeptide, a TIM3 binding polypeptide, another checkpoint inhibitor binding polypeptide, a neoantigen polypeptide or a combination thereof. Other target binding polypeptides and combinations described above and hereafter can be suitable.

The carrier can comprise a human serum albumin (HSA) or a variant thereof. Other carriers, such as BSA, describe above and hereafter can also be suitable.

The capturing device of this invention can be configured to capture one or more target substances comprising a protein, a chemical agent, a toxin, a drug, bacteria, a virus, virus nucleic acids, virus protein, a particle, an aggregate or a combination thereof.

The capturing device of this invention is free from moving parts. As mentioned above, the immobile phase of the capturing device remains stationary when the body fluid is passing through the affinity matrix. The entire capturing device is produced in one unit and eliminates the need for additional components, such as a motor for providing motions.

The capturing device can further comprise at least one coupling device for coupling the capturing device with a dialysis device selected from a hemodialysis device or a peritoneal dialysis device, wherein the body fluid is directed to flow through the capturing device and the dialysis device tandemly or parallelly. Examples of configurations are schematically illustrated in FIG. 5-FIG. 7. An input splitting device (29) and a joining device (30) (FIG. 7) can be suitable. Another example of a configuration is schematically illustrated in FIG. 8.

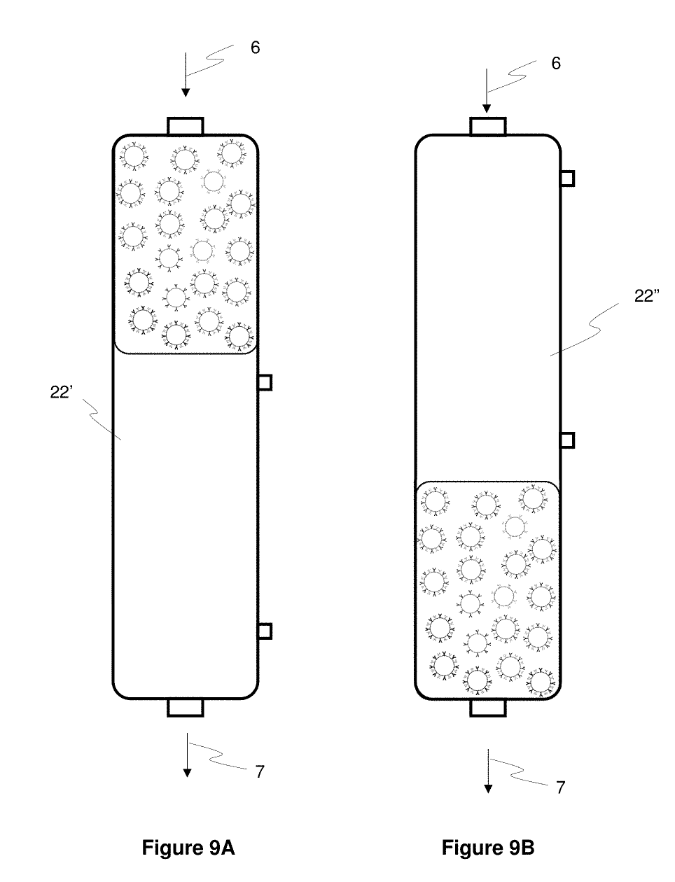

The capturing device and the dialysis device can be configured into a single capturing-dialysis hybrid device. In one example of a configuration, a hybrid device (22′) is configured so that a capturing device and a dialysis device share a common intake to receive the body fluid, wherein the body fluid first flows through the capturing device, then exits and flows through the dialysis device (FIG. 9A). In another example of a configuration, a hybrid device (22″) is configured so that a capturing device and a dialysis device share a common outlet to output a processed body fluid, wherein the body fluid first flows through the dialysis device, then exits and flows through the capturing device (FIG. 9B). Such hybrid device can provide a convenient use when all of the body fluid is desired to go through the capturing and the dialysis devices. When only a part of a body fluid is desired to pass the capturing device, a parallel configuration, such as the one illustrated in FIG. 7 can be preferred.

This invention is further directed to a method for treating a medical condition in a subject in need thereof, the method comprising:

directing a body fluid of the subject into a capturing device, wherein the body fluid contains at least one target substance related to the medical condition, and wherein the capturing device comprises an immobile phase and at least one affinity agent affixed to the immobile phase, the affinity agent is configured to bind to the target substance;

passing the body fluid through the capturing device for the affinity agent to bind to the target substance and immobilize at least a portion of the target substance on the immobile phase, producing a processed body fluid having at least a portion of the target substance removed therefrom; and

directing the processed body fluid back to the subject.

The body fluid can be aqueous humour and vitreous humour (fluid in eyes), bile, blood, blood plasma, blood serum, gastric juice, lymph or a combination thereof.

The medical condition can comprise a cancer, a lung disease, a liver disease, a kidney disease, diabetes, a blood disease, a pancreatic disease, sepsis, viral infection, drug overdose, intoxication, or a combination thereof. In one example, one or more proteins, such as PD-L1 or CTLA-4 can be removed from patients suffering from a cancer or autoimmune diseases. In another example, an opioid overdose patient can be treated by using an opioid receptor as an affinity agent to remove opioids such as dezocine, morphine, fentanyl, butorphanol, nalbuphine, or a combination thereof from the blood. In yet another example, a patient with severe sepsis that is not responsive to antibiotics can be treated with bacteria binding agents as the affinity agent using the method of this invention to remove bacteria from blood.

The method of this invention can further comprise directing the body fluid through a dialysis device selected from a hemodialysis device or a peritoneal dialysis device, wherein the body fluid is directed to flow through the capturing device and the dialysis device tandemly or parallelly. The system configurations exemplified here, such as those illustrated in FIG. 5-FIG. 8, can be suitable.

The body fluid can be directed at a flow rate in a range of from 20 ml/min to 250 ml/min passing through the capturing device.

The method of this invention can further comprise measuring a pre-concentration of the target substance in the body fluid before passing through the capturing device, a post-concentration of the target substance in the processed body fluid, or a combination thereof.

Suitable for the method of this invention, the affinity agent can comprise a biological affinity agent, a chemical affinity agent, a physical affinity agent, or a combination thereof, wherein the biological affinity agent comprises a protein, an antibody, a binding fragment of an antibody, an antigen, a fragment of an antigen, a neoantigen polypeptide comprising one or more neoantigens or epitopes, an antagonist, a receptor, nucleotides, deoxynucleotides, thiolated nucleotides, a binding fragment of a receptor, a bacteria-binding protein, a virus-binding protein, an opioid receptor, a toxin-binding protein, a fusion protein comprising a target binding polypeptide linked to a carrier or a combination thereof. The antibody comprising an anti-PD-1 antibody, an anti-PD-L1 antibody, an anti-PD-L2 antibody, an anti-CTLA-4 antibody, an anti-LAG-3 antibody, an anti-TIM3 antibody, or a combination thereof, can be suitable.

Suitable for the method of this invention, the fusion protein can be produced by a production process comprising expressing a fusion coding region comprising codes encoding the target binding polypeptide and the carrier in an expression host. Aforementioned expression vectors, express systems, purification and modifications can be suitable.

Suitable for the method of this invention, the target binding polypeptide can comprise a PD-1 binding polypeptide, a PD-L1 binding polypeptide, a PD-L2 binding polypeptide, a CTLA-4 binding polypeptide, a LAG-3 binding polypeptide, a TIM3 binding polypeptide, another checkpoint inhibitor binding polypeptide, a neoantigen polypeptide, or a combination thereof.

Suitable for the method of this invention disclosed herein, the carrier can comprise a human serum albumin (HSA) or a functional variant as described above and hereafter. Other carriers mentioned above can also be suitable.

As mentioned above, the target substance can comprise a protein, a chemical agent, a toxin, a drug, bacteria, a virus, virus nucleic acids, virus protein, a particle, an aggregate or a combination thereof.

The method of this invention can further comprise the steps of measuring a pre-concentration of at least one tumor marker in a body fluid before passing through the capturing device, a post-concentration of a tumor marker in a processed body fluid, or a combination thereof. The tumor marker can include, but not limited to, CA15-3 (breast cancer), CA19-9 antigen (gastrointestinal tumors), CA-125 (ovarian cancers), PSA (prostate cancers), CEA (carcinoembryonic antigen), or a combination thereof.

One advantage of this invention is to provide an improved or an alternative treatment to patients who suffer from PD-1/PD-L1 immunotherapy related AKI that may require dialysis and may not be able to continue the PD-1/PD-L1 immunotherapy. The process of this invention provides an alternative approach to reduce PD-1/PD-L1 level in patient's system that can help T-cell activation for fighting cancer. Due to its higher affinity to PD-1 versus PD-L1, PD-L2 binding polypeptide or PD-L2 antibody can also be suitable as an affinity agent to deplete PD-1 proteins or PD-1 bearing tumor cells in the body fluid.

This invention is further directed to a pharmaceutical composition comprising a fusion protein comprising at least one target binding polypeptides selected from a PD-1 binding polypeptide, a PD-L1 binding polypeptide, a PD-L2 binding polypeptide, a CTLA-4 binding polypeptide, a LAG-3 binding polypeptide, a TIM3 binding polypeptide, another checkpoint inhibitor binding polypeptide, or a neoantigen polypeptide. In one example, the fusion protein can comprise at least two of target binding polypeptides selected from a PD-1 binding polypeptide, a PD-L1 binding polypeptide, a PD-L2 binding polypeptide, a CTLA-4 binding polypeptide, a LAG-3 binding polypeptide, a TIM3 binding polypeptide, another checkpoint inhibitor binding polypeptide, or a neoantigen polypeptide. In another example, the fusion protein can comprise a PD-1 binding polypeptide and at least one of CTLA-4 binding polypeptide, LAG-3 binding polypeptide, TIM3 binding polypeptide, another checkpoint inhibitor binding polypeptide, or neoantigen polypeptide. In yet another example, the fusion protein can comprise a PD-L1 binding polypeptide and at least one of CTLA-4 binding polypeptide, LAG-3 binding polypeptide, TIM3 binding polypeptide, another checkpoint inhibitor binding polypeptide, or neoantigen polypeptide. In yet another example, the fusion protein can comprise one or more PD-L1 binding polypeptides and one or more PD-1 binding polypeptides. PD-L1 binding polypeptides bind to different part of a PD-L1 protein can be suitable. PD-1 binding polypeptides bind to different part of a PD-1 protein can be suitable.

The fusion protein of the pharmaceutical composition can further comprise a carrier that comprises a human serum albumin (HSA) or a functional variant thereof. Aforementioned HSA, BSA, variants thereof, or a combination thereof, can be suitable.

Immobilized matrix, such as membranes or beads are used for removing molecules from blood in treating some medical conditions, such as clinically diagnosed dialysis-related amyloidosis (DRA) (Abe et al., Kidney International, Vol. 64, pp. 1522-1528, 2003). In March 2015, Lixelle® Beta 2-microglobulin (β₂M) Apheresis Column (Kaneka Pharma America LLC, New York, N.Y. 10036, USA, under respective trademark) was approved by U.S. Food and Drug Administration (FDA) for treating DRA in patients in the USA. However, such matrix can only adsorb hydrophobic proteins, such as β₂M, by binding with the hydrophobic hexadecyl hydrocarbonate group. Other devices, such as the one described in U.S. Pat. No. 6,099,732, granted on Aug. 8, 2000, requires rotation of cylinders and electric motors. Such rotation may introduce undesired risks of damaging blood cells.

This invention provides a simple way for depletion or partial depletion of PD-L1 protein in body fluid of a cancer patient, such as in blood. The PD-L1 protein can be a soluble PD-L1 (sPD-L1) or a membrane bound PD-L1 (mPD-L1). Depletion conditions can be configured to only deplete sPD-L1 protein in blood and not the cells that have mPD-L1 on their membranes. Depletion conditions can also be configured to deplete both sPD-L1 and mPD-L1 by capturing T cells having mPD-L1 on their surfaces. Reduction of sPD-L1 level has been shown to increase survival time for cancer patients. Not wishing to be bound by a particular theory or mechanism, it is believed that reduced level of sPD-L1 in blood may enhance efficacy of anti-PD-1 or anti-PD-L1 immunotherapy since less anti-PD-L1 antibodies become neutralized by sPD-L1 in blood leading to more anti-PD-L1 antibody available for binding to PD-1 on T cells. This may lead to reduced dosage requirement for anti-PD-L1 antibody and subsequently reduced toxicity side effects, such as the acute kidney injury (AKI). The process of this invention can also provide an alternative treatment to cancer patients who suffer complications of the anti-PD-1 /anti-PD-L1 immunotherapy, such as AKI. Further, reduction of PD-1 or PD-L1 levels in blood may lead to changes in PD-1/PD-L1 equilibrium on T cells or tumor-T cell interactions that may have impact of the T-cell inhibition induced by cancer cells. Reduction of other target substances, such as CTLA-4 or sCTLA-4 may help to treat or obliviate some conditions such as autoimmune diseases.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

### Example 1: Anti-PD-1 Antibody Affinity Beads

Anti-human PD-1 antibody and Protein A beads can be obtained from Invitrogen/ThermoFisher. The antibody is affixed to the Protein beads according to conventional process. The antibody-coated beads are then blocked with HSA to block all Protein A binding sites on the beads that are not occupied by the antibody to produce the anti-PD-1 affinity beads. The anti-PD-1 affinity beads are then packed into a column coupled with an intake and an outlet. The beads can be optionally dried before packed into the column.

### Example 2: Anti-PD-L1 Antibody Affinity Beads

Anti-PD-L1 affinity beads and a column are produced according to the same procedure described in Example 1, but with an anti-human PD-L1 monoclonal antibody.

### Example 3: Anti-PD-1 and Anti-PD-L1 Antibodies Affinity Beads

Anti-PD-1 and anti-PD-L1 affinity beads and a column are produced according to the same procedure described in Example 1, but with an anti-human PD-1 monoclonal antibody and an anti-human PD-L1 monoclonal antibody.

### Example 4: Fusion Protein Affinity Beads

A fusion protein comprising HSA-linker1-PD-1 binding polypeptide (αPD-1)-linker2-PD-L1 binding polypeptide (αPD-L1) is constructed by joining an HSA coding sequence together with the αPD-1 and αPD-L1 sequences derived from monoclonal antibodies based on Lee, et al. (Nature Communications, 7:13354, Oct. 31, 2016. DOI: 10.1038/ncomms13354). The fusion protein can be expressed in E. coli and purified. The purified fusion protein can be affixed to beads according to the procedure described above. The fusion protein beads can be packed into a column.

1. A process for depleting at least one target substance from a body fluid of a subject, said process comprising the steps of:
directing said body fluid of said subject into a capturing device, wherein said body fluid contains said target substance, and wherein said capturing device comprises an affinity matrix comprising an immobile phase and at least one affinity agent affixed to said immobile phase, said affinity agent is configured to bind to said target substance; and
passing said body fluid through said affinity matrix for said affinity agent to bind and immobilize said target substance to said immobile phase, producing a processed body fluid having at least a portion of said target substance depleted therefrom.

2. The process of claim 1, wherein said affinity agent comprises a biological affinity agent, a chemical affinity agent, a physical affinity agent, or a combination thereof, wherein said biological affinity agent comprises a protein, an antibody, a binding fragment of an antibody, an antigen, a fragment of an antigen, a neoantigen polypeptide comprising one or more neoantigens or epitopes, an antagonist, a receptor, nucleotides, deoxynucleotides, thiolated nucleotides, a binding fragment of a receptor, a bacteria-binding protein, a virus-binding protein, a toxin-binding protein, a fusion protein comprising a target binding polypeptide linked to a carrier, or a combination thereof.

3. The process of claim 2, wherein said antibody comprises an anti-PD-1 antibody, an anti-PD-L1 antibody, an anti-PD-L2 antibody, an anti-CTLA-4 antibody, an anti-LAG-3 antibody, an anti-TIM3 antibody or a combination thereof.

4. The process of claim 2, wherein said fusion protein is produced by a production process comprising expressing a fusion coding region comprising codes encoding said target binding polypeptide and said carrier in an expression host.

5. The process of claim 4, wherein said target binding polypeptide comprises a PD-1 binding polypeptide, a PD-L1 binding polypeptide, aPD-L2 binding polypeptide, a CTLA-4 binding polypeptide, a LAG-3 binding polypeptide, a TIM3 binding polypeptide, a checkpoint inhibitor binding polypeptide, a neoantigen polypeptide or a combination thereof.

6. The process of claim 4, wherein said carrier comprises a human serum albumin (HSA) or a functional variant thereof.

7. The process of claim 1, wherein said target substance has a molecular weight of greater than 10 KDa and wherein said target substance comprises a protein, a chemical agent, a toxin, a drug, bacteria, a virus, virus nucleic acids, virus protein, a particle, an aggregate or a combination thereof.

8. The process of claim 1, wherein said body fluid is aqueous humour, vitreous humour, bile, blood, blood plasma, blood serum, gastric juice, lymph or a combination thereof.

9. The process of claim 1 further comprising directing the body fluid through a dialysis device selected from a hemodialysis device or a peritoneal dialysis device, wherein said body fluid is directed to flow through said capturing device and said dialysis device tandemly or parallelly.

10. The process of claim 1, wherein said body fluid is directed at a flow rate in a range of from 1 ml/min to 250 ml/min passing through said capturing device.

11. The process of claim 1 further comprising measuring a pre-concentration of said target substance in said body fluid before passing through said capturing device, a post-concentration of said target substance in said processed body fluid, or a combination thereof.

12. The process of claim 1 further comprising the step of directing said processed body fluid back to said subject.

13. A capturing device for depleting at least one target substance from a body fluid, said device comprising:
An affinity matrix having an immobile phase having at least one affinity agent affixed thereon, said affinity agent is configured to bind to said target substance and immobilize said target substance on said immobile phase; and
a device housing to house said affinity matrix, said device housing comprises a housing intake for receiving said body fluid into the device housing, a fluid passage for containing said affinity matrix and a housing outlet, said fluid passage is functionally coupled to said housing intake and said housing outlet;
wherein said capturing device is configured to receive said body fluid through said housing intake, pass said body fluid through said fluid passage and output a processed said body fluid from said housing outlet.

14. The capturing device of claim 13, wherein said immobile phase is selected from beads, membrane, porous matrix, scaffold, or a combination thereof.

15. The capturing device of claim 13, wherein said affinity agent comprises a biological affinity agent, a chemical affinity agent, a physical affinity agent, or a combination thereof, wherein said biological affinity agent comprises a protein, an antibody, a binding fragment of an antibody, an antigen, a fragment of an antigen, a neoantigen polypeptide comprising one or more neoantigens or epitopes, an antagonist, a receptor, nucleotides, deoxynucleotides, thiolated nucleotides, a binding fragment of a receptor, a bacteria-binding protein, a virus-binding protein, a toxin-binding protein, an opioid receptor, a fusion protein comprising a target binding polypeptide linked to a carrier or a combination thereof.

16. The capturing device of claim 15, wherein said antibody comprises an anti-PD-1 antibody, an anti-PD-L1 antibody, an anti-PD-L2 antibody, an anti-CTLA-4 antibody, an anti-LAG-3 antibody, an anti-TIM3 antibody or a combination thereof.

17. (canceled)

18. The capturing device of claim 15, wherein said target binding polypeptide comprises a PD-1 binding polypeptide, a PD-L1 binding polypeptide, a PD-L2 binding polypeptide, a CTLA-4 binding polypeptide, a LAG-3 binding polypeptide, a TIM3 binding polypeptide, a checkpoint inhibitor binding polypeptide, a neoantigen polypeptide or a combination thereof.

19. (canceled)

20. The capturing device of claim 13, wherein said target substance comprises a protein, a chemical agent, a toxin, a drug, bacteria, a virus, virus nucleic acids, virus protein, a particle, an aggregate or a combination thereof

21. The capturing device of claim 13 further comprising at least one coupling device for coupling said capturing device with a dialysis device selected from a hemodialysis device or a peritoneal dialysis device, wherein said body fluid is directed to flow through said capturing device and said dialysis device tandemly or parallelly.

22. The capturing device of claim 21, wherein said capturing device and said dialysis device are configured into a capturing-dialysis hybrid device.

23-41. (canceled)
